(19) United States
(12) Patent Application Publication
(10) Pub. No.: US 2026/0351348 A1
(43) Pub. Date: Nov. 5, 2026

(54) COMPUTATIONAL PROTEIN ENGINEERING VIA LANGUAGE MODEL-GUIDED RECOMBINATION AND IMMUNOGENICITY OPTIMIZATION

(71) Applicant: Massachusetts Institute of Technology, Cambridge, MA (US)

(72) Inventors: Manvitha Ponnapati, Cambridge, MA (US); Sapna Sinha, Providence, RI (US); Edward Boyden, Chestnut Hill, MA (US); Joseph Jacobson, Brookline, MA (US)

(73) Assignee: Massachusetts Institute of Technology, Cambridge, MA (US)

(21) Appl. No.: 19/659,276

(22) Filed: Apr. 27, 2026

Related U.S. Application Data
(60) Provisional application No. 63/794,433, filed on Apr. 25, 2025.

(51) Int. Cl. G16B 35/10; A61K 38/00; C07K 14/405; C40B 40/10; G16B 40/00

(52) U.S. Cl. CPC G16B 35/10; C07K 14/405; G16B 40/00; A61K 38/00; C07K 2319/00; C40B 40/10

(57) ABSTRACT

A data-driven method for designing recombinant protein libraries utilizes protein language models to identify optimal crossover sites directly from sequence information. Log-likelihood profiles are spline-smoothed to locate maximal transition points, which serve as recombination boundaries. Sequence blocks are swapped via Markov Chain Monte Carlo sampling informed by multiple sequence alignments and evolutionary context to yield diverse chimeric proteins with maintained function and low structural disruption. Direct Preference Optimization finetunes the language model to penalize MHC Class I epitope motifs, enabling selection of variants with decreased immunogenic potential while preserving function. The approach permits rapid, structure-independent generation of therapeutic and industrial protein variants and encompasses related systems and computer-readable media.

### CROSS-REFERENCE TO RELATED APPLICATION

This application claims the benefit of priority of U.S. provisional application No. 63/794,433, filed Apr. 25, 2025, the contents of which are herein incorporated by reference.

### SEQUENCE LISTING

The Sequence Listing XML, an electronic file having the file name “Sequence_Listing_61790-115np_MIT.xml,” which was created on May 27, 2026, and has a size of 2,386 bytes, is hereby incorporated by reference in its entirety pursuant to 37 CFR 1.835 (a) (2).

### FIELD OF THE INVENTION

The present invention relates generally to protein engineering and, more particularly, to computational methods for designing recombinant protein libraries that exhibit reduced structural disruption and lowered immunogenicity by leveraging protein language models, spline-based crossover site identification, Markov Chain Monte Carlo (MCMC) sampling, and policy optimization like Direct Preference Optimization (DPO).

### BACKGROUND OF THE INVENTION

Protein recombination has long been employed in protein engineering to diversify protein sequences while maintaining function, with the goal that new variants may exhibit enhanced properties or additional beneficial characteristics. SCHEMA (Voigt et al., 2002; Mateljak et al., 2019), a structure-guided method, identifies optimal recombination sites by minimizing disruption to protein contact maps. A contact between residues i and j is considered disrupted if those amino acids do not co-occur in any parent sequence. While effective, SCHEMA requires high-resolution structural data and computational analysis of all residue-residue contacts, limiting its applicability.

More recently, generative deep-learning approaches including inverse folding methods and structure-based design can create de novo proteins (Anishchenko et al., 2021; Dauparas et al., 2022; Ruffolo et al., 2024; Hayes et al., 2024). These methods generate diverse protein sequences by inverse sampling from structures, scaffolding active sites, and leveraging structure prediction models like AlphaFold (Jumper et al., 2021). Candidates are filtered using metrics such as pLDDT, TM-score, and others (Bryant et al., 2022; Zhang & Skolnick, 2004). However, these metrics correlate poorly with protein function due to structural degeneracy, where similar structures arise from diverse sequences with varying functionality (Alley et al., 2019).

Protein language models (PLMs) such as ESM2 and MSATransformer capture evolutionary information from multiple sequence alignments (MSAs), encoding functional constraints without requiring structural data (Rives et al., 2021; Rao et al., 2021). Recent work has shown that PLMs can identify functionally important regions and guide protein design. For example, Ponnapati et al. (2025) demonstrated that abrupt transitions in average log-likelihood scores from ESM2, when represented as splines, correlate with optimal crossover sites identified by SCHEMA, eliminating the need for structure information.

For therapeutic applications, minimizing immunogenicity is critical. Multiple mechanisms trigger immune responses, including MHC Class I binding, TAP transport, proteolytic cleavage, and T-cell recognition (Neefjes et al., 2011). Limited labeled immunogenicity data restricts application of sequence-based diffusion models. However, reinforcement learning approaches like Direct Preference Optimization (DPO) enable alignment of PLMs toward specific design goals using preference datasets (Widatalla et al., 2024; Olivecrona et al., 2017). DPO has been successfully applied to protein stability optimization and can generalize beyond observed mutational landscapes (Widatalla et al., 2024).

As can be seen, there is a need for methods that combine evolutionary information from protein language models with structure-minimizing recombination strategies and targeted optimization for therapeutic properties such as reduced immunogenicity, without requiring extensive structural data or large training datasets.

### SUMMARY OF THE INVENTION

In one aspect of the present invention, a chimeric protein comprises recombined sequence blocks from at least two parent proteins, wherein the protein exhibits at least 70% functional activity of a parent protein, has at least 50% reduction in predicted MHC Class I binding epitopes compared to the parent protein, and preserves functional residues. The chimeric protein may be a channelrhodopsin exhibiting at least 80% photocurrent activity with at least 70% reduction in predicted HLA-A*02:01 epitopes. The invention further encompasses isolated nucleic acids encoding such proteins, pharmaceutical compositions comprising the proteins, and recombinant protein libraries.

In another aspect of the present invention, a method for designing recombinant proteins with reduced immunogenicity comprises: obtaining at least one parent protein sequence; computationally identifying crossover sites using evolutionary information from a pretrained protein language model; generating chimeric protein sequences by computationally recombining sequence blocks at the crossover sites; computationally optimizing the chimeric sequences to reduce predicted immunogenic epitopes while preserving functional residues; selecting chimeric sequences predicted to exhibit maintained protein function and reduced immunogenicity; and producing at least one selected chimeric protein by expressing the sequence in a biological expression system.

In a further aspect, the method comprises computing per-residue log-likelihood scores using a protein language model, fitting a continuous spline to the scores to generate a smoothed importance function, identifying local maxima as optimal crossover sites, applying Markov Chain Monte Carlo sampling to generate chimeric sequences by recombining blocks at the crossover sites, using policy optimization such as Direct Preference Optimization to align the protein language model for reducing MHC Class I epitope binding, selecting sequences satisfying optimization objectives, and producing recombinant proteins by expressing selected sequences in a host cell. The method identifies crossover sites without requiring structural data, produces diverse libraries with low structural disruption, and integrates preference-based optimization to selectively reduce predicted T-cell epitopes for therapeutic applications.

These and other features, aspects and advantages of the present invention will become better understood with reference to the following drawings, description, and claims.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A is a scatter plot comparing disruption factor versus mutation distance for chimeric sequences generated using SCHEMA crossover sites versus CAMP spline-identified crossover sites according to an embodiment of a method the present disclosure.

FIG. 1B is a structural diagram showing crossover sites determined by prior art SCHEMA RASPP for PDB ID 1G68.

FIG. 1C is a structural diagram showing crossover sites determined using the spline method for the same protein as FIG. 1B.

FIG. 1D is a graph showing spline fit to log-likelihood scores from ESM2 for according to an embodiment of the present disclosure.

FIG. 1E is a scatter plot showing disruption factor versus mutation distance trade-off for channelrhodopsin according to an embodiment of the present disclosure.

FIG. 2A is a scatter plot comparing disruption factor versus sequence diversity for chimeras generated using SCHEMA-RASPP crossover sites with MCMC sampling.

FIG. 2B is a scatter plot comparing disruption factor versus sequence diversity for chimeras generated using log-likelihood-based crossover sites with MCMC sampling according to the FIG. 1A embodiment.

FIG. 3A is an annotated protein sequence showing channelrhodopsin C1C2 with functional residues, spline-identified crossover points, and strong and weak HLA-A*02:01 binding 9-mer peptides identified.

FIG. 3B is a bar chart comparing fraction of masked epitopes sampled as non-binders between pretrained and DPO-aligned models according to embodiments of the present disclosure.

FIG. 3C is a bar chart comparing total number of MHC binders generated by pretrained versus DPO-aligned models according to the embodiments of FIG. 3B.

FIG. 3D is a three-dimensional protein structure diagram of channelrhodopsin (PDB ID: 3UG9) with functional regions highlighted.

FIG. 4A is a scatter plot showing TM-score versus mutations for Beta Lactamase sequences using methods according to embodiments of the present disclosure.

FIG. 4B is a scatter plot showing TM-score versus mutations for sequences generated from esmGFP's MSA using recombination methods according to embodiments of the present disclosure.

FIG. 5 is a schematic diagram of a computational workflow according to embodiments of the present disclosure.

### DETAILED DESCRIPTION OF THE INVENTION

The following detailed description is of the best currently contemplated modes of carrying out exemplary embodiments of the invention. The description is not to be taken in a limiting sense but is made merely for the purpose of illustrating the general principles of the invention, since the scope of the invention is best defined by the appended claims.

Broadly, one embodiment of the present invention is a computational method for designing recombinant protein libraries with reduced structural disruption and lowered immunogenicity by integrating protein language models (PLMs), spline-based crossover site identification, Markov Chain Monte Carlo (MCMC) sampling, and Direct Preference Optimization (DPO).

Unless otherwise defined, all technical and scientific terms used herein have the same meaning as commonly understood by one of ordinary skill in the art. Singular forms “a,” “an,” and “the” include plural referents unless the context clearly dictates otherwise. All publications and patent applications cited herein are incorporated by reference in their entirety for all purposes.

All publications, patents, and patent applications cited herein are hereby incorporated by reference in their entirety for all purposes.

The methods of the present disclosure identify crossover sites from protein language model (PLM) log-likelihood profiles without structural data, enabling application to proteins lacking crystal structures. PLMs are neural networks trained on large protein sequence datasets that predict amino acid probabilities at each position based on sequence context. By combining MCMC sampling—a probabilistic method that iteratively proposes and scores sequence modifications—with MSA-conditioned language models, the methods achieve greater sequence diversity at equivalent disruption levels compared to structure-based approaches like SCHEMA. Multiple sequence alignments (MSAs) are collections of evolutionarily related proteins aligned to show corresponding positions across homologs.

The methods execute on computing systems with processors, memory, storage, and preferably GPUs. The system receives parent protein sequences, typically comprising a wild-type target plus 2-4 homologous variants that serve as starting material for generating chimeric libraries-collections of recombinant proteins combining sequence blocks from multiple parents.

The system runs a pretrained PLM such as ESM-2 (trained on 250 million sequences) or MSATransformer (trained on MSAs to incorporate evolutionary context) to compute per-residue log-likelihood. At position j, the PLM computes probabilities p_j(a) for all 20 amino acids:

[symbol]

_j=(1/|A|)Σ_(a∈A)log p_j(a)
where A is the amino acid alphabet and p_j(a) is the probability of amino acid a at position j. The log-likelihood [symbol] _j averages these log probabilities: [symbol] _j=(1/20)×sum of log p_j(a). Higher (less negative) values indicate positions where many amino acids are plausible, suggesting evolutionary flexibility.

A cubic spline S(x) is fitted to these discrete scores, creating a continuous importance function representing smoothed evolutionary plausibility at each position. Local maxima (where S′(x)=0 and S″(x)<0) identify crossover sites-positions where recombination between parent sequences minimizes structural disruption.

MCMC sampling then iteratively: (1) selects a crossover site C_i, (2) swaps the adjacent block with one sampled from the MSA, (3) scores the candidate using perplexity=exp (−Score/L) where Score=−Σ_t log p(amino acid_t|context), and (4) accepts or rejects via the Metropolis-Hastings algorithm, which always accepts improved scores and probabilistically accepts worse scores based on the difference. The Score sums negative log probabilities for each amino acid given sequence context-higher scores indicate less plausible sequences. Perplexity normalizes by sequence length L, with lower values (5-10) indicating plausible sequences and higher values (>15) indicating implausible ones. This process generates chimeric libraries combining blocks from different parents.

In some embodiments, Direct Preference Optimization (DPO) fine-tunes the PLM using paired training examples to bias toward sequences with desired properties. The training dataset D={(x⁺_i, x⁻_i)} comprises sequence pairs where x⁺ contains few MHC Class I epitopes (9-amino-acid peptides that bind immune receptors and trigger T-cell responses) and x⁻ contains many epitopes. The model learns to assign higher probability to x⁺, biasing it toward low-immunogenicity sequences.

Training minimizes loss L_i=−log σ(Δ_i) where Δ_i=log π_θ(x⁺_i)−log π_0 (x⁻_i) and σ is the sigmoid function. Here, Δ_i measures the difference between the model's log-probabilities for preferred versus dispreferred sequences, and the loss penalizes cases where the model assigns higher probability to high-epitope sequences. The sigmoid function converts preference differences to a 0-1 probability scale. Minimizing this loss biases the DPO-aligned PLM toward generating sequences with reduced immunogenicity.

Candidates are filtered using multiple metrics: AlphaFold2 pLDDT scores (>70 for reliable structural predictions), where pLDDT is a per-residue confidence metric (0-100) indicating predicted positional accuracy; active residue preservation; ESM2 perplexity; phylogenetic distance; functional residue topology measured by RMSD (root mean square deviation between protein conformations after optimal superposition); and netMHCPan epitope scoring, which predicts peptide binding to MHC receptors. The methods may be embodied as non-transitory computer-readable media, deployed locally, or provided as cloud API services.

The DPO framework described herein is adaptable to optimization objectives beyond MHC Class I epitope reduction. For each target property, preference datasets are constructed by identifying sequence features correlated with the desired trait and generating (x⁺, x⁻) pairs where x⁺ exhibits superior performance. Multi-allele immunogenicity optimization expands the preference dataset to penalize binding across HLA-A02:01, HLA-A01:01, HLA-B*07:02, and other common alleles. Additional properties amenable to this approach include MHC Class II binding (using 15-mer predictions from NetMHCIIpan), thermostability (using predicted ΔΔG or melting temperatures), protease resistance (identifying and penalizing cleavage motifs), solubility (using aggregation propensity scores from tools like CamSol or Aggrescan), and other biophysical or functional attributes for which predictive models or scoring functions exist.

When optimizing multiple properties simultaneously, multi-objective algorithms such as weighted sum methods or Pareto optimization identify sequences balancing competing objectives. The MCMC sampler can be biased toward sequences with improved properties by incorporating additional scoring terms into the acceptance criterion, weighting perplexity alongside property-specific predictions.

Selection may employ machine learning models trained on experimental protein stability data to predict folding success, expression yield, or functional activity from sequence alone, supplementing the structural and immunogenicity filters described above.

Recombinant proteins generated by the disclosed methods have utility in therapeutic and diagnostic applications where immunogenicity poses clinical risks. Biologic drugs including therapeutic enzymes, monoclonal antibodies, and gene therapy vectors can elicit anti-drug antibodies (ADAs) that reduce efficacy or cause adverse reactions. By systematically reducing MHC Class I epitope burden while preserving function, the DPO-aligned variants exhibit lower predicted immunogenicity. For example, channelrhodopsin variants with reduced HLA-A*02:01 epitopes are suitable for optogenetic gene therapies with decreased risk of immune rejection. Similarly, enzyme replacement therapies (e.g., β-lactamase variants) benefit from extended serum half-life when immunogenic epitopes are eliminated. Therapeutic compositions may comprise the recombinant proteins formulated with pharmaceutically acceptable carriers for parenteral, topical, or other routes of administration.

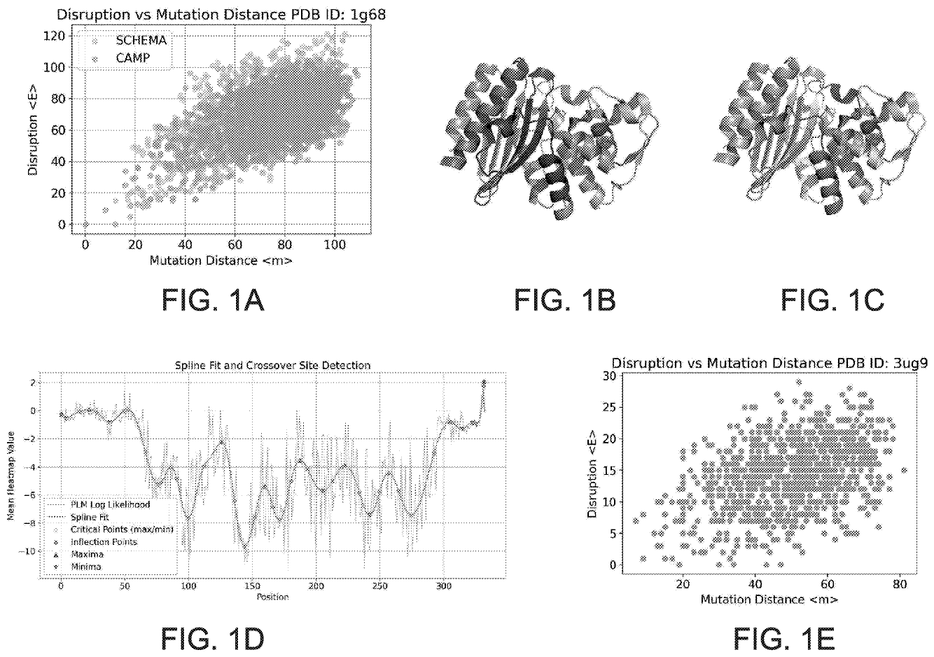

Referring to FIGS. 1A-1E, 2A-2B, 3A-3D, 4A-4B, and 5, experimental method validation results and a pipeline schematic are illustrated.

FIGS. 1A-1E validate the spline-based crossover identification method against prior art SCHEMA. FIG. 1A compares disruption factor E versus mutation distance m for chimeric sequences generated from PDB ID 1G68, with green points representing sequences using SCHEMA-identified crossover sites and purple points representing sequences using CAMP spline-identified sites. The comparison demonstrates that CAMP achieves similar disruption factors with reduced mutation distance from parent sequences. FIG. 1B shows the protein structure of 1G68 with crossover sites identified by SCHEMA-RASPP marked on the structure. FIG. 1C shows the same protein with crossover sites identified by the spline method, demonstrating comparable spatial positioning to SCHEMA sites. FIG. 1D illustrates the spline fitting process, where a light green line represents raw per-residue PLM log-likelihood scores, the light purple line represents the fitted cubic spline function S(x), yellow circles indicate critical points (max/min), pink circles indicate inflection points, and dark purple triangles indicate local maxima, and dark green triangles indicate local minima, with local maxima (where S′(x)=0 and S″(x)<0) identified as candidate crossover sites. FIG. 1E plots disruption factor versus mutation distance for channelrhodopsin (PDB ID: 3UG9), confirming the favorable trade-off between structural preservation and sequence diversity achieved by spline-identified crossover sites.

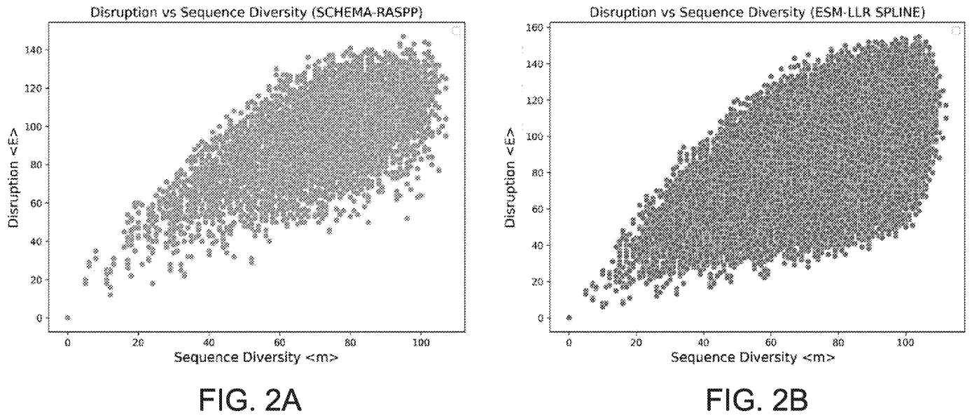

FIGS. 2A-2B compare sequence space exploration using MCMC sampling. FIG. 2A plots disruption factor E versus sequence diversity for chimeras generated using SCHEMA-RASPP crossover sites (green points). FIG. 2B shows the same analysis for chimeras generated using the log-likelihood-based spline method (purple points), demonstrating that the spline method produces substantially more samples in the high-diversity, low-disruption region of sequence space.

### EXAMPLES

### Example 1: Channelrhodopsin C1C2 Immunogenicity Reduction

Wild-type chimeric C1C2 opsin (PDB ID: 3UG9) and two homologous opsins were used as parent sequences. The 315-residue reference sequence is:
STGSDATVPVATQDGPDYVFHRAHERMLFQTSYTLENNGSVICIPNNG QCFCLAWLKSNGTNAEKLAANILQWITFALSALCLMFYGYQTWKSTCGWEEIYVA TIEMIKFIIEYFHEFDEPAVIYSSNGNKTVWLRYAEWLLTCPVILIHLSNLTGLANDY NKRTMGLLVSDIGTIIVWGTTAALSKGYVRVIFFLMGLCYGIYTFFNAAKVYIEAYH TVPKGRCRQVVTGMAWLFFVSWGMFPILFILGPEGFGVLSVYGSTVGHTIIDLMS KNCWGLLGHYLRVLIHEHILIHGDIRKTTKLNIGGTEIEVETLVEDEAEAGAVSSED LYFQ

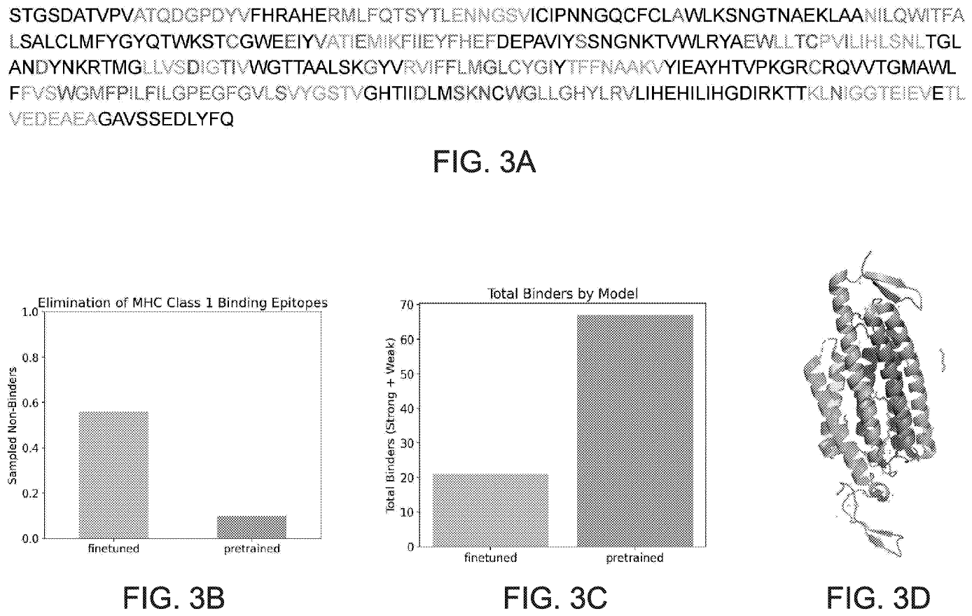

Functional residues were held constant during sampling as detailed in Table 1. Referring to FIG. 3A, the annotated sequence shows functional residues (red), spline-identified crossover points (blue), strong HLA-A*02:01 binding 9-mer peptides (purple), and weak binders (green).

TABLE 1
Functional Constraints for Channelrhodopsin C1C2

| Residue | | |
|---|---|---|
| Category | Residues Held Constant | Reason |
| Pore Residues | His134, His265, Glu82, | Ion conductance |
| | Glu83, Asn258, Glu90, | |
| | Glu97, Glu101, Gln56 | |
| Retinal Binding | Ser155, Thr159, Gly181, | Chromophore binding |
| Pocket | Asp156, Cys128, Asp253, | and photocycle |
| | Glu123, Lys93, Gly163 | |
| Flexible Loops | N-terminus (49-83), | Low AlphaFold2 |
| | C-terminus (319-342) | confidence for |
| | | disordered regions |

Seven local maxima were identified via spline fitting, corresponding to positions between structural domains.

The MCMC sampler was run with parameters T=1000 steps per round and N=20 independent rounds. A total of 10,000 sequences were generated with 2,300 (23%) passing the filter criteria of disruption E≤0.35 and perplexity≤12. The sequences that passed the filter criteria exhibited the following. Mean Disruption E of 0.26 indicates chimeric sequences maintain approximately 74% of parent protein contacts intact, representing relatively low structural perturbation. The mutation range of 12-95 substitutions shows sequence diversity spans from conservative variants to highly divergent chimeras. Mean pLDDT of 78 indicates AlphaFold2 predicts reliable structures for the library, exceeding the 70-point threshold for confident predictions.

A preference dataset comprising 500 preferred (low-epitope) and 500 dispreferred (high-epitope) sequence pairs was constructed. Epitopes were identified using netMHCPan-4.1 for the HLA-A*02:01 allele. Each masked epitope region generated 100 candidate samples, which were classified by predicted binding strength. Twenty percent of epitope mask locations were held out for validation. Performance metrics are detailed in Table 2. Referring to FIGS. 3B-3C, the DPO-aligned model achieved an 84% reduction in strong binder regeneration and a 70% reduction in total binders compared to the baseline pretrained model. FIG. 3D displays the three-dimensional channelrhodopsin structure with functional regions highlighted.

TABLE 2
DPO Immunogenicity Reduction Performance

| | Masked | Masked | | |
|---|---|---|---|---|
| | Epitopes | Epitopes | Total | |
| | as Strong | as Non- | Binders | Reduction VS. |
| Model | Binders | Binders | Generated | Baseline |
| Baseline | ~55% | ~45% | ~65 | — |
| MSATransformer | | | | |
| DPO-Aligned | ~12% | ~88% | ~20 | 84% fewer |
| MSATransformer | | | | strong binders, |
| | | | | 70% fewer |
| | | | | total binders |

The properties of the selected lead candidate are summarized in Table 3.

TABLE 3
Lead Candidate CHR-DPO-42 Properties

| Property | Value |
|---|---|
| Disruption Factor E | 0.22 |
| Mutations from Wild-Type | 58 |
| ESM2 Perplexity | 8.3 |
| Strong HLA-A*02:01 Binders | 3 (vs. 19 in wild-type) |
| Mean pLDDT | 82 |
| Photocurrent vs. Wild-Type | Within 5% |

### Example 2: TEM-1 β-Lactamase Functional Diversification

Wild-type TEM-1 β-lactamase and four homologous sequences were used with an MSA comprising 150 sequences sharing greater than 40% identity. Catalytic residues listed in Table 4 were held constant throughout sampling.

TABLE 4
Functional Constraints for TEM-1 β-Lactamase

| Residue Category | Residues Held Constant | Reason |
|---|---|---|
| Catalytic Triad | Ser70, Lys73, Ser130, Glu166 | β-lactam hydrolysis |

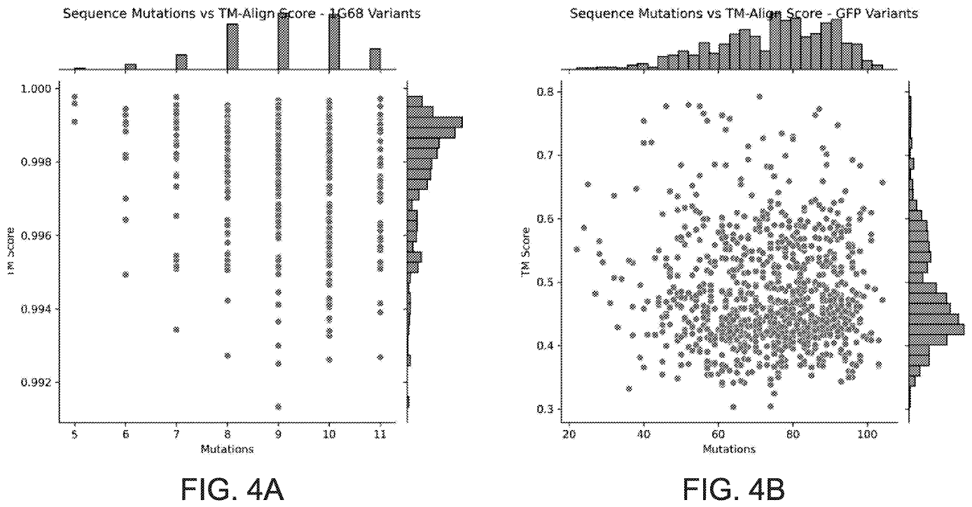

Nine crossover sites were identified. Of 8,500 total sequences, 1,200 (14%) passed filter criteria of disruption E≤0.40 and perplexity≤15. Mutation distances ranged from 25-95 substitutions with mean disruption 0.31, TM-align scores exceeding 0.95, and mean pLDDT greater than 75. Referring to FIG. 4A, the scatter plot of TM-score (0.992-1.000) versus mutations (5-11) for Beta Lactamase sequences (red points) demonstrates high structural conservation despite sequence variation.

### Example 3: esmGFP Validation

The esmGFP sequence with a natural GFP homolog MSA was used for method validation. Six crossover sites were identified and 5,000 sequences were generated. Referring to FIG. 4B, the scatter plot of TM-score (0.4-0.8) versus mutations (20-100) for esmGFP variants (green points) demonstrates the structural and sequence diversity achievable by the recombination method. Several generated sequences matched known functional GFP variants, validating that PLM-identified crossover sites effectively partition protein sequences for functional recombination.

### Example 4: Molecular Dynamics Validation

To validate structural predictions, selected chimeric sequences are subjected to molecular dynamics simulations using GROMACS with the AMBER99SB force field. Proteins are solvated, energy-minimized, equilibrated (NVT then NPT), and simulated for 100 ns. Structural stability is assessed via RMSD, RMSF, radius of gyration, and secondary structure retention. Channelrhodopsin variant CHR-DPO-42 exhibits mean backbone RMSD of 0.23 nm with stable Rg and 94% secondary structure retention, confirming the disruption factor threshold effectively identifies MD-stable sequences. Sequences with disruption E>0.40 show RMSD>0.5 nm and progressive unfolding.

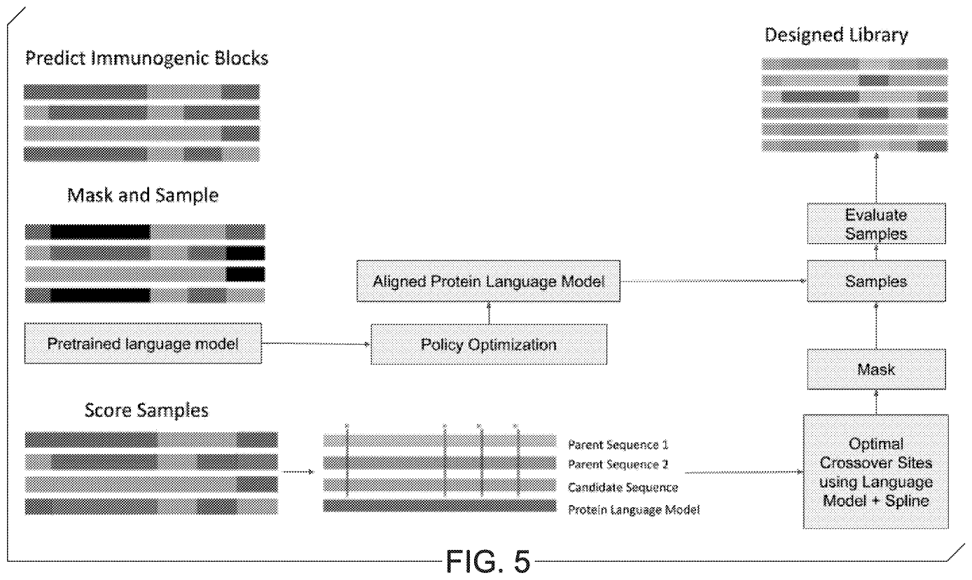

Methods execute on cloud or local hardware with GPUs for PLM inference. FIG. 5 is a schematic diagram illustrating the CAMP computational workflow. The left side shows training data generation: immunogenic blocks are predicted and highlighted in protein sequences, regions are masked and sampled to create variants, a pretrained language model generates candidates, and samples are scored for epitope content to create preference pairs. The center shows the DPO training process: the pretrained language model undergoes policy optimization using the preference dataset to produce an aligned protein language model. The right side shows library generation: parent sequences are profiled using the language model, optimal crossover sites are identified via spline analysis, the aligned model generates samples with masked regions at crossover sites, samples are evaluated for structural and functional metrics, and the final designed library is output. Arrows indicate data flow between components.

It should be understood, of course, that the foregoing relates to exemplary embodiments of the invention and that modifications may be made without departing from the spirit and scope of the invention as set forth in the following claims.

What is claimed is:

1. A chimeric protein comprising recombined sequence blocks from at least two parent protein sequences, wherein:
(a) the chimeric protein exhibits at least 70% functional activity of a parent protein;
(b) the chimeric protein has at least 50% reduction in predicted MHC Class I binding epitopes compared to the parent protein; and
(c) functional residues of the parent protein sequences are preserved in the chimeric protein.

2. The chimeric protein of claim 1, wherein the protein is a channelrhodopsin having recombined blocks from channelrhodopsin C1C2 and related opsin homologs, wherein:
(a) the protein exhibits at least 80% photocurrent activity of wild-type C1C2;
(b) the protein has at least 70% reduction in predicted HLA-A*02:01 binding epitopes compared to wild-type C1C2; and
(c) functional residues at positions His134, His265, Glu82, Glu83, Asn258, Glu90, Glu97, Glu101, Gln56, Ser155, Thr159, Gly181, Asp156, Cys128, Asp253, Glu123, Lys93, and Gly163 are preserved.

3. An isolated nucleic acid encoding the chimeric protein of claim 1.

4. A pharmaceutical composition comprising the chimeric protein of claim 1 and a pharmaceutically acceptable carrier.

5. A recombinant protein library comprising at least 100 chimeric proteins of claim 1.

6. A method for designing recombinant proteins with reduced immunogenicity, comprising:
(a) obtaining at least one parent protein sequence;
(b) computationally identifying crossover sites in said protein sequence using evolutionary information from a pretrained protein language model;
(c) generating chimeric protein sequences by computationally recombining sequence blocks at the crossover sites;
(d) computationally optimizing the chimeric sequences to reduce predicted immunogenic epitopes while preserving functional residues;
(e) selecting chimeric sequences predicted to exhibit maintained protein function and reduced immunogenicity; and
(f) producing at least one selected chimeric protein by expressing the sequence in a biological expression system.

7. The method of claim 6, wherein computationally identifying crossover sites comprises computing sequence-based scores for residue positions using the protein language model and identifying positions of high evolutionary flexibility as crossover sites.

8. The method of claim 6, wherein generating chimeric protein sequences comprises probabilistic sampling conditioned on evolutionary context from a multiple sequence alignment.

9. The method of claim 6, wherein computationally optimizing comprises training the protein language model using preference datasets comprising sequence pairs with differential predicted immunogenicity.

10. A method for designing recombinant protein libraries, comprising:
(a) obtaining at least one parent protein sequence;
(b) computing log-likelihood scores for each residue position in said protein sequence using a protein language model;
(c) fitting a continuous spline to the log-likelihood scores to generate a smoothed importance function;
(d) identifying local maxima of the smoothed importance function as optimal crossover sites for protein recombination;
(e) applying Markov Chain Monte Carlo sampling to generate chimeric protein sequences by recombining sequence blocks at said crossover sites;
(f) using policy optimization to align a protein language model for reducing MHC Class I epitope binding in the chimeric sequences;
(g) selecting chimeric protein sequences with maintained function and predicted to satisfy at least one optimization objective; and
(h) producing at least one recombinant protein by expressing a protein sequence selected in step (g) in a host cell.

11. The method of claim 10, wherein computing log-likelihood scores in step (b) comprises calculating, for each residue position j: [symbol] _j=(1/|A|) Σ_(a∈A) log p_j(a) where A is an amino acid alphabet and p_j(a) is a probability assigned by the protein language model to amino acid a at position j.

12. The method of claim 10, wherein the Markov Chain Monte Carlo sampling in step (e) comprises:
(i) performing T sampling steps per round for N independent rounds;
(ii) at each timestep, randomly selecting a crossover site and replacing one adjacent sequence block with a block sampled from a multiple sequence alignment;
(iii) evaluating candidate sequences using protein language model perplexity; and
(iv) clustering sequences based on similarity after each round and selecting cluster representatives as seeds for subsequent rounds.

13. The method of claim 10, wherein the policy optimization in step (f) comprises:
(i) generating a preference dataset comprising paired sequences (x⁺, x⁻) where x⁺ exhibits reduced MHC Class I epitope binding compared to x⁻;
(ii) computing a preference score Δ=log π_0 (x⁺)−log π_0 (x⁻);
(iii) computing a loss function L=−log σ(Δ) where σ is a sigmoid function; and
(iv) updating model parameters to minimize the loss function.

14. The method of claim 13, wherein generating the preference dataset comprises:
(a) identifying MHC Class I binding epitopes using netMHCPan;
(b) masking identified epitope regions;
(c) sampling multiple candidate sequences at masked regions using the protein language model;
(d) classifying candidates as preferred (x⁺) or dispreferred (x⁻) based on predicted MHC binding affinity; and
(e) forming preference pairs from classified candidates.

15. The method of claim 10, wherein functional residues are held constant during recombination, wherein functional residues are selected from active site residues, binding pocket residues, catalytic residues, and structurally essential residues.

16. The method of claim 15, wherein the protein is a channelrhodopsin and the functional residues comprise pore residues selected from His134, His265, Glu82, Glu83, Asn258, Glu90, Glu97, Glu101, and Gln56, and retinal binding pocket residues selected from Ser155, Thr159, Gly181, Asp156, Cys128, Asp253, Glu123, Lys93, and Gly163.

17. The method of claim 10, wherein selecting chimeric protein sequences in step (g) comprises filtering based on:
(i) disruption factor below 0.35;
(ii) protein language model perplexity below 12;
(iii) AlphaFold predicted pLDDT score above 70; and
(iv) number of predicted MHC Class I binding epitopes representing at least 50% reduction compared to a parent sequence.

18. The method of claim 10, wherein the protein language model in step (b) is ESM2, and the protein language model used for policy optimization in step (f) is ESM-MSA-1b.

19. The method of claim 10, wherein the MHC Class I epitopes are 9-mer peptides predicted to bind to HLA-A*02:01 with a percentile rank below 0.5 as determined by netMHCPan-4.1.

20. The method of claim 10, wherein selecting chimeric protein sequences in step (g) further comprises selecting for at least one property selected from: reduced MHC Class II epitope binding, increased thermostability, increased protease resistance, increased solubility, reduced aggregation propensity, and increased protein expression yield.


SEQUENCE LISTING (1 sequence)

SEQ ID NO: 1 - protein, 334 aa, synthetic construct
STGSDATVPVATQDGPDYVFHRAHERMLFQTSYTLENNGSVICIPNNGQCFCLAWLKSNGTNAEKLAANILQWITFALSALCLMFYGYQTWKSTCGWEEIYVATIEMIKFIIEYFHEFDEPAVIYSSNGNKTVWLRYAEWLLTCPVILIHLSNLTGLANDYNKRTMGLLVSDIGTIIVWGTTAALSKGYVRVIFFLMGLCYGIYTFFNAAKVYIEAYHTVPKGRCRQVVTGMAWLFFVSWGMFPILFILGPEGFGVLSVYGSTVGHTIIDLMSKNCWGLLGHYLRVLIHEHILIHGDIRKTTKLNIGGTEIEVETLVEDEAEAGAVSSEDLYFQ